(19) United States
(12) Plant Patent Application Publication
(10) Pub. No.: US 2016/0309631 P1
(43) Pub. Date: Oct. 20, 2016

(54) Male pistachio variety named 'FAMOSO'

(50) ;

(71) Applicant: The Regents of the University of California, Oakland, CA (US)

(72) Inventors: Craig E. KALLSEN, Davis, CA (US); Dan E. PARFITT, Davis, CA (US); Joseph MARANTO, Davis, CA (US)

(21) Appl. No.: 14/999,156

(22) Filed: Apr. 4, 2016

Related U.S. Application Data
(60) Provisional application No. 62/147,540, filed on Apr. 14, 2015.

(51) Int. Cl. A01H 5/00

(52) U.S. Cl. PLT/152

(57) ABSTRACT

A new male pistachio variety (Pistacia vera L.) designated as ‘Famoso’ is provided that is well-suited for serving as a pollenizer of the female pistachio variety ‘Kerman’ and other mid-to late-season pistachio varieties. ‘Famoso’ has a better overlap in the flowering period of ‘Kerman’ than does the industry-standard male pistachio variety ‘Peters’. ‘Famoso’ is also more precocious than ‘Peters’; ‘Famoso’ produces flowers three to four years after budding as compared to ‘Peters’, which typically takes four to five years to produce flowers after budding. ‘Famoso’ also has a flowering period that is more coincident with the flowering period of ‘Kerman’, while ‘Peters’ often flowers later than ‘Kerman’ and has been reported to have poor blooms in some years.

### CROSS-REFERENCE TO RELATED APPLICATION

This application claims the benefit of U.S. Provisional Application No. 62/147,540, filed Apr. 14, 2015, which is incorporated herein by reference in its entirety.

### LATIN NAME

Botanical/commercial classification: (Pistacia vera L.), new pistachio cultivar.

### VARIETAL DENOMINATION

The varietal denomination of the claimed pistachio variety is ‘Famoso’.

### BACKGROUND OF THE INVENTION

An objective of pistachio breeding programs is to develop improved male pollinators of the female pistachio variety ‘Kerman’. The female pistachio variety ‘Kerman’ is the main later-season pistachio cultivar grown in California and in other parts of the world. The male pistachio variety ‘Peters’ is a common male cultivar used as a pollen source to pollinate ‘Kerman’. Although ‘Peters’ is viewed as the industry standard pollinator variety for ‘Kerman’, ‘Peters’ often flowers later than ‘Kerman’ and has a history of providing poor flowering overlap with ‘Kerman’ in some years, especially in years with insufficient chilling. Even in seasons of relatively adequate chilling, ‘Peters’ is generally not very precocious. ‘Peters’ is slow to produce flowers as a juvenile tree as compared to ‘Kerman’. The female ‘Kerman’, at 6^{th} or 7^{th} leaf, will have many flowers, while ‘Peters’ trees will have none or relatively few. Typically, the female variety ‘Kerman’ is ˜one year ahead of the male variety ‘Peters’ in terms of flower development, which results in a year of lost production. Further, in low chill years, ‘Peters’ has performed very poorly. In some young orchards, ‘Peters’ produced almost no flowers, the flowers that were produced had no pollen, and it often bloomed 1-2 weeks later than ‘Kerman’. This has resulted in inadequate pollination of ‘Kerman’ and reduced yield potential.

Pistachio growers are in need of a male pistachio variety that is more precocious than ‘Peters’, performs better in low chill years, and has a better flowering overlap with the female variety ‘Kerman’. Thus, there exists a need for improved pollinator varieties, such as male varieties having improved flowering overlap with ‘Kerman’. The present male pistachio variety ‘Famoso’ described herein is a product of the breeding efforts to produce improved male pistachio varieties.

### SUMMARY OF THE INVENTION

The present invention relates to a new and distinct pistachio cultivar (Pistacia vera L.) that has been denominated as ‘Famoso’, and more particularly as a male pistachio variety that has a better overlap with the flowering period of the female pistachio variety ‘Kerman’ than does the male pistachio variety ‘Peters’. The male pistachio variety ‘Famoso’ is more precocious than ‘Peters’; ‘Famoso’ produces flowers three to four years after budding as compared to ‘Peters’, which typically takes four to five years to produce flowers after budding. The male pistachio variety ‘Famoso’ also has a flowering period that is more coincident with the flowering period of the female pistachio variety ‘Kerman’, while the male pistachio variety ‘Peters’ often flowers later than ‘Kerman’ and has been recorded to have poor flowering overlap with ‘Kerman’ and to have poor blooms during some years, especially during years with low chilling, which are expected to become more frequent in future years in view of the warming climate.

By providing a better overlap in flowering period with ‘Kerman’ than is provided by ‘Peters’, ‘Famoso’ should improve ‘Kerman’ yield, especially in years when ‘Peters’ flowers significantly later than ‘Kerman’. ‘Famoso’ flowering is more consistent than ‘Peters’, and ‘Famoso’ also produces a large amount of pollen over an extended period. ‘Famoso’ is potentially significant as an alternative to ‘Peters’ for the California, New Mexican, Arizona, and world-wide pistachio industry. ‘Famoso’ may be used as the pollenizer for mid- to late-season pistachio cultivars such as ‘Kerman’. ‘Famoso’ may also be used as an additional pollenizer of female pistachio varieties (e.g. ‘Kerman’) in orchards along with the male variety ‘Peters’ or other male pistachio varieties.

The cross that produced ‘Famoso’ was originally made during Year 1, and the original seedling of ‘Famoso’ was planted at a research plot near Bakersfield, Calif. during Year 2. The cross was made between Pistacia vera L. female ‘2-35’ and Pistacia vera L. male ‘ES#4’. ‘Famoso’ was originally designated as selection ‘B19-69’. Following initial identification, selection ‘B19-69’ was subsequently planted in field tests plots near Famoso, Calif. and other locations (e.g. Little Creek and Tejon Ranch). Selection ‘B19-69’ was asexually reproduced during Year 11 when buds from this selection were budded to rootstocks, which were then planted during Year 11 at two test locations in California, USA. Specifically, ‘B19-69’ was asexually reproduced in the trial using grafting (T-buds) directly onto pre-planted rootstocks in the two trial areas. The buds were collected from the U.C. seedling selection trial located in Kern County, Calif., USA. Selection ‘B19-69’ was chosen as a candidate for release under the variety name ‘Famoso’.

### BRIEF DESCRIPTION OF THE DRAWINGS

FIG. 1 illustrates ‘Famoso’ in bloom during Year 25.

FIG. 2A illustrates flower clusters of ‘Famoso’ just prior to dehiscence. FIG. 2B illustrates flower clusters of ‘Famoso’ at anther dehiscence.

FIG. 3A illustrates the trunk, branches, and canopy of ‘Famoso’. FIG. 3B illustrates the leaves of ‘Famoso’

FIG. 4A illustrates the mean germination ratio of freshly-collected pollen for ‘Famoso’ and other male pistachio varieties in Year 26. Shown is the analysis of means (MiniTab) with 5% SD, where a value of 1.0=100%. FIG. 4B illustrates a fitted means plot of the germination data presented in FIG. 4A.

FIG. 5A illustrates the mean germination ratio of stored pollen for ‘Famoso’ and other male pistachio varieties in Year 26. Shown is the analysis of means (MiniTab) with 5% SD, where a value of 1.0=100%. FIG. 5B illustrates boxplots of viable pollen ratios by variety.

FIG. 6A illustrates the mean pollen weight per inflorescence (grams) for ‘Famoso’ and other male pistachio varieties in Year 26. Shown is the analysis of means (MiniTab) with 5% SD. FIG. 6B illustrates boxplots of pollen weight by variety.

FIG. 7A-FIG. 7B illustrates a comparison of inflorescence density for ‘Famoso’ (FIG. 7A) and ‘Peters’ (FIG. 7B).

### BOTANICAL DESCRIPTION OF THE PLANT

The following is a detailed botanical description of the new male pistachio cultivar designated as ‘Famoso’, including the key differentiating characteristics of this variety and comparisons of certain characteristics of ‘Famoso’ to other pistachio varieties.

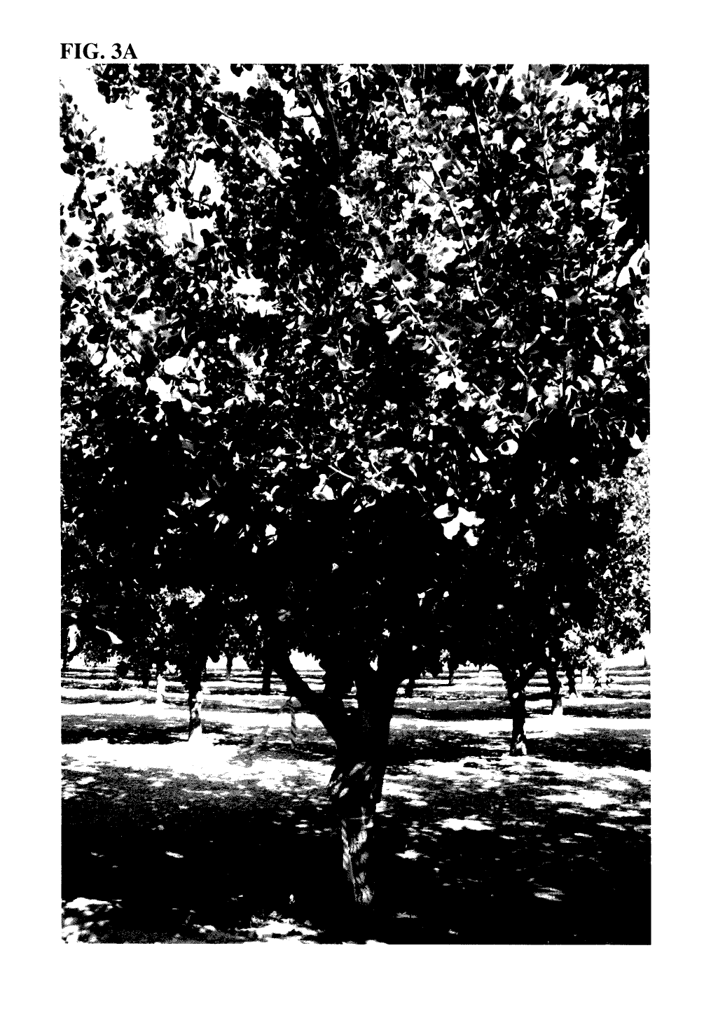

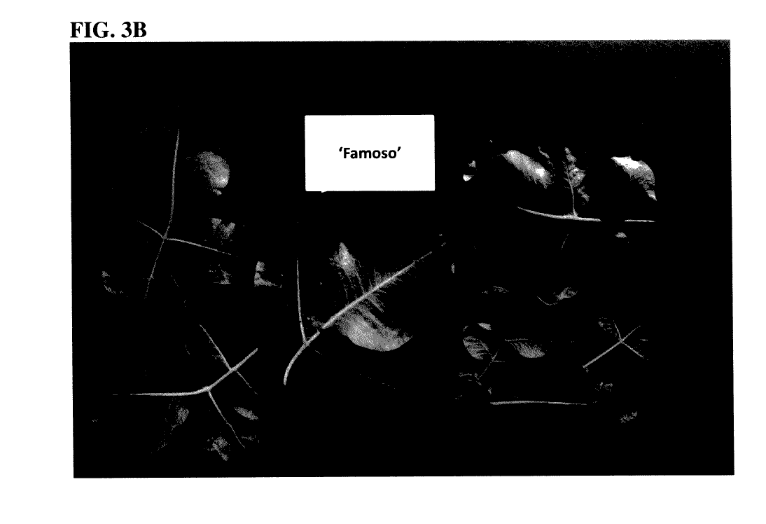

Color of leaves: For leaves, color evaluations were done on at least 3 leaves, each new and mature, collected at random from a ‘Famoso’ tree during the summer of Year 25. Color descriptions are based on the color standards presented in Royal Horticultural Society; R.H.S. colour chart v. 3 QC495 R8 V.3. Mature leaves, top surface=137B, bottom surface=137B, 137C, new leaves, top surface=137A, 137B, bottom surface=139C, 138B, leaf midrib=145C. An image of the leaves of ‘Famoso’ is presented in FIG. 3B.

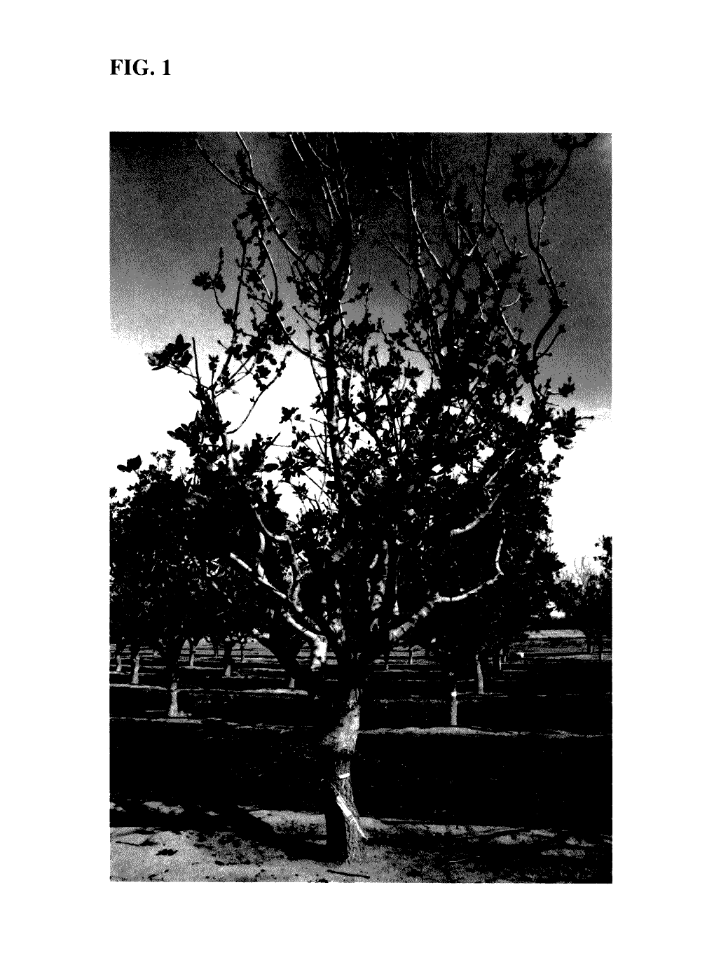

Flowering Time: ‘Famoso’ is at full bloom ˜1.4 days before or after ‘Kerman’ and 7-9 days after ‘Golden Hills’, but 2-7 days before ‘Peters’ and ˜4 days after ‘Randy’ (depending on the season). The male pistachio variety ‘Famoso’ flowered at approximately the same time as the female pistachio variety ‘Kerman’ over a five year period at two locations in the major pistachio production areas of the San Joaquin valley (TABLE 1A). TABLE 1A shows mean “full bloom” flowering dates for ‘Famoso’ and ‘Randy’ over a 5 year period, referenced to ‘Kerman’ (day 0). The pistachio trees described in TABLE 1A were established (grafted into the orchard) during Year 13. Overall, ‘Randy’ flowered about 4 days before ‘Famoso’, while ‘Famoso’ peak flowering was about 1.5 days ahead of ‘Kerman’. An image of ‘Famoso’ in bloom is presented in FIG. 1. An image of a ‘Famoso’ tree, including images of the trunk, branches, and canopy, is presented in FIG. 3A.

TABLE 1A
Flowering time of ‘Famoso’ and other varieties relative to ‘Kerman’

| | Tejon Ranch | | | | | Little Creek (Famoso) | | | | |
|---|---|---|---|---|---|---|---|---|---|---|
| | 4/5, | 4/13 | 4/21, | 4/21, | 4/20, | 4/1, | 4/13, | 4/19, | 4/16, | 4/17, |
| Date^{a} | Year 18 | Year 19 | Year 20 | Year 21 | Year 22 | Year 18 | Year 19 | Year 20 | Year 21 | Year 22 |
| | Flowering Date vs. ‘Kerman’ (female) | | | | | Flowering Date vs. ‘Kerman’ (female) | | | | |
| ‘Kerman’ | 0 | 0 | 0 | 0 | 0 | 0 | 0 | 0 | 0 | 0 |
| ‘Famoso’ | 0 | −2 | −3 | −3 | −2 | −1 | −1 | 0 | −2 | −1 |
| ‘Randy’ | −4 | −5 | −6 | −7 | −5 | 0 | −9 | −6 | −7 | −4 |
| | Mean Flowering Date vs. 'Kerman' (both locations) | | | | | | | | | |
| ‘Kerman’ | 0 | | | | | | | | | |
| ‘Famoso’ | −1.5 | | | | | | | | | |
| ‘Randy’ | −5.3 | | | | | | | | | |
| | Flower Density Score^{b} | | | | | Flower Density Score^{b} | | | | |
| ‘Famoso’ | 3 | 3 | 2 | 3 | 3 | 2 | 3 | 3 | 4 | 3 |
| ‘Randy’ | 4 | 4 | 4 | 4 | 4 | 3 | 4 | 4 | 4 | 4 |

| | |
|---|---|
| | Mean Flower Density Score (both locations) |
| ‘Famoso’ | 2.9 |
| ‘Randy’ | 3.9 |

^{a}This is the date that the female variety ‘Kerman’ flowered in the year identified.
^{b}Flower density presented from 1 = low to 4 = high.

Low Chill Flowering: TABLE 1B presents flowering time results for Year 26, which was a low chill year. In Year 26, ‘Famoso’ was similar to ‘Randy’ in terms of flowering date, and ‘Famoso’ flowered long before ‘Peters’. Both ‘Randy’ and ‘Famoso’ overlapped with ‘Kerman’. Despite the overlap between ‘Randy’ and ‘Kerman’ in this low chill year, from TABLE 1A above, it is seen that, in a normal chill year, ‘Randy’ would flower too early for good pollenization of ‘Kerman’. ‘Famoso’ is intermediate to ‘Randy’ and ‘Peters’ for flowering date and thus can overlap with ‘Kerman’ in both higher and lower chilling seasons.

TABLE 1B
Year 26 flowering evaluation of ‘Famoso’.
Note mid-bloom (3)/full bloom (4) dates for overlap.

| | | Year 26 Evaluation Date: | | | | | |
|---|---|---|---|---|---|---|---|
| | | March | March | March | March | March | April |
| Cultivar | | 16 | 19 | 23 | 26 | 30 | 2 |
| Famoso | Male | 1.9 | 2.3 | 2.6 | 2.7 | 3.3 | 6 |
| Randy | Male | 2 | 2.4 | 2.5 | 2.6 | 3.5 | 6 |
| Peters | Male | 1 | 0.9 | 1 | 1.3 | 2.6 | 2.8 |
| Kerman | Female | 2 | 2.5 | 2.9 | 4.5 | 6 | 6 |
| Golden | Female | 2.4 | 3.2 | 3.5 | 6 | 6 | 6 |
| Hills | | | | | | | |
| Lost | Female | 2.4 | 3.2 | 4.2 | 6 | 6 | 6 |
| Hills | | | | | | | |

Bloom ratings 0 to 6:
0 = dormant buds,
1 = green tip on flower bud,
2 = > (greater than) 5 open buds on tree,
3 = mid bloom,
4 = full bloom,
5 = late bloom,
6 = bloom finished

Flower Density: On a flower density scale (1=low, 4=high), ‘Famoso’ ranks at an average of ˜3. This is higher than the rating of ‘Peters’, which ranks at a maximum of 2 as a mature tree. ‘Randy’ ranks as a strong 4.

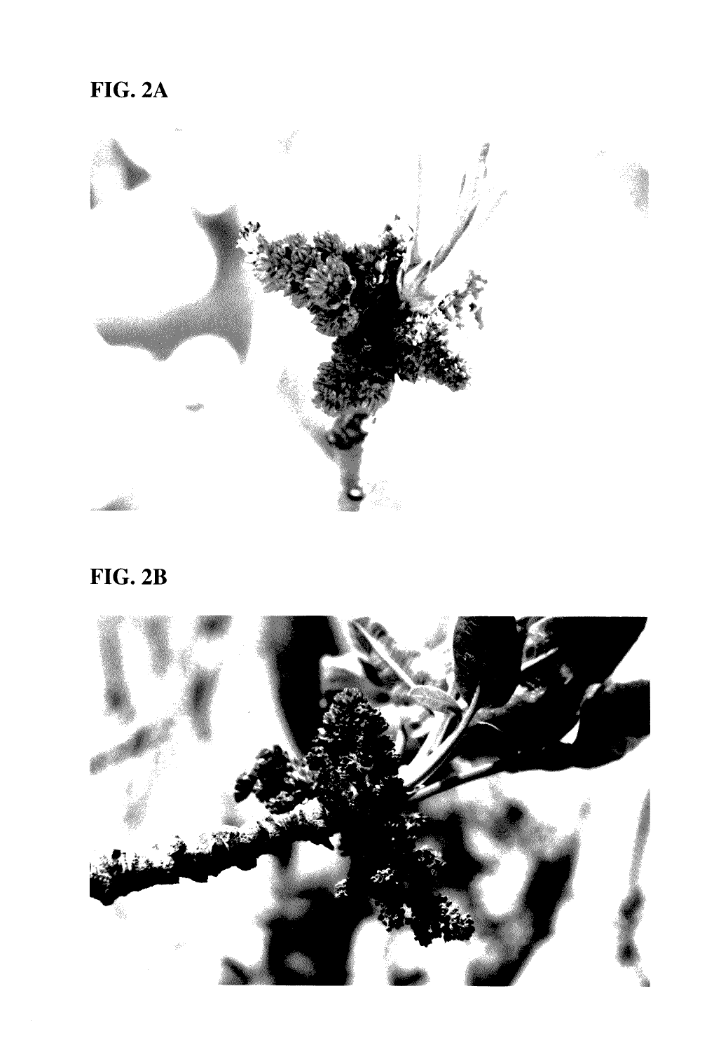

Pollen: ‘Famoso’ has good pollen viability, providing good quantities of viable pollen. ‘Famoso’ sheds a large amount of pollen over a reasonably extended period (19, 14 and 20 days at the Famoso trial in Year 20, Year 21, and Year 22, respectively), and has a highly coincident flowering period with ‘Kerman’. Pollen germination at pollen shed for ‘Famoso’ is high and similar to both ‘Randy’ and ‘Peters’. ‘Famoso’ produces more abundant flowers and pollen as a juvenile tree compared to ‘Peters’. Views of ‘Famoso’ flower clusters both prior to and at dehiscence are presented in FIG. 2A and FIG. 2B. Pollen counts (germination) for ‘Famoso’ taken directly after pollen collection in Year 25 was similar to ‘Randy’ but less than ‘Peters’ (TABLE 2). The pistachio trees described in TABLE 2 were established (grafted into the orchard) during Year 13, and would thus be 12 years old (pollen count data collected in Year 25). ‘Famoso’ should be a superior pollinizer for ‘Kerman’ due to better overlap of pollen shed with the ‘Kerman’ bloom period, especially when the orchard first comes into bearing.

TABLE 2
Pollen counts taken directly after pollen collection in Year 25

| | | Mean | Std. dev. |
|---|---|---|---|
| Variety | n | % | % |
| ‘Famoso’ | 8 | 69.711 | 8.796 |
| ‘Peters’ | 6 | 79.034 | 8.014 |
| ‘Randy’ | 8 | 67.752 | 7.179 |

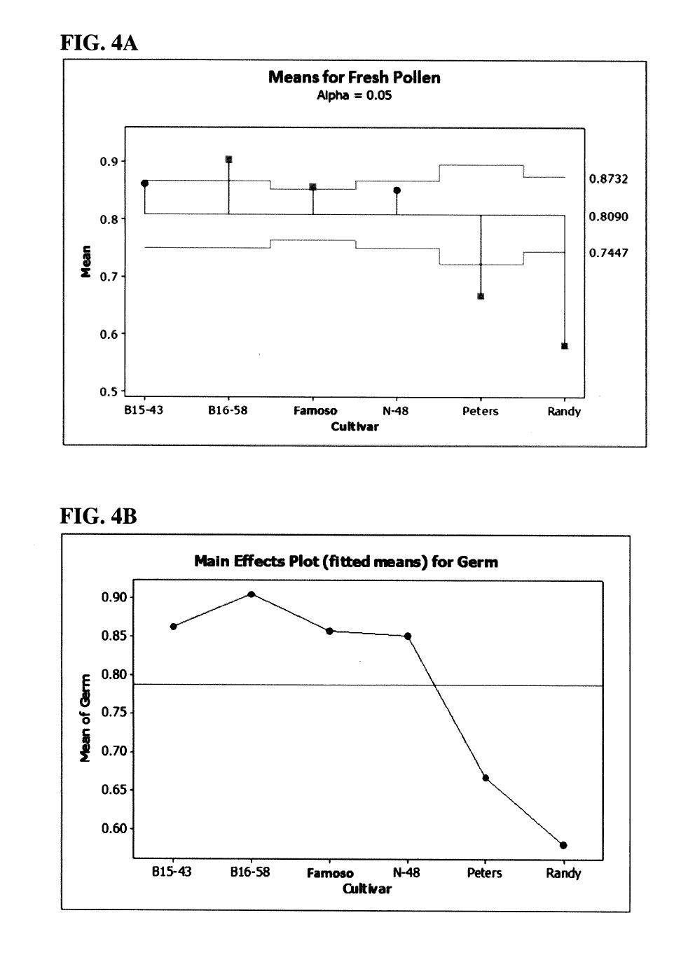

Pollen Germination (Year 26): Pollen germination is somewhat a snapshot in time, as it can vary from early to late bloom. Different inflorescences and flowers on the same branch will shed pollen at different times during the bloom period. This is a semi-random process. To analyze fresh pollen counts (germination) in Year 26, pollen from a variety of male pistachio varieties was collected between March 21 and March 31. Pollen was germinated on 18% sucrose with some boron and calcium nitrate, and at low light in a humid chamber. The pollen of each cultivar was germinated using hanging drop slides. Each slide had two wells and three of these slides were prepared for each cultivar. Germination results are presented below. ‘Randy’ is a proven pollinizer for the 30,000+ acres of ‘Golden Hills’ planted in the San Joaquin Valley, and ‘Famoso’ had higher pollen germination percentages than ‘Randy’ or ‘Peters’ in this study (Year 26)(TABLE 3A, TABLE 3B, FIG. 4A, and FIG. 4B).

TABLE 3A
Least Squares Means for germination of fresh pollen in Year 26,
counted immediately after flowering.

| Cultivar | Mean % | SE Mean % |
|---|---|---|
| N-48 | 85.09 | 2.334 |
| Peters | 66.67 | 3.301 |
| Randy | 58.09 | 2.557 |
| B15-43 | 86.19 | 2.334 |
| B16-58 | 90.40 | 2.334 |
| Famoso | 85.66 | 1.906 |

TABLE 3B
GLM ANOVA showing highly significant differences among cultivars.
GLM Analysis of Variance for Germination of Fresh Pollen

| Source | DF | SS | MS | F | P |
|---|---|---|---|---|---|
| Cultivar | 5 | 0.84564 | 0.16913 | 25.87 | 0.000 |
| Error | 64 | 0.41841 | 0.00654 | | |
| Total | 69 | 1.26405 | | | |

S = 0.0808560
R-Sq = 66.90%
R-Sq(adj) = 64.31%

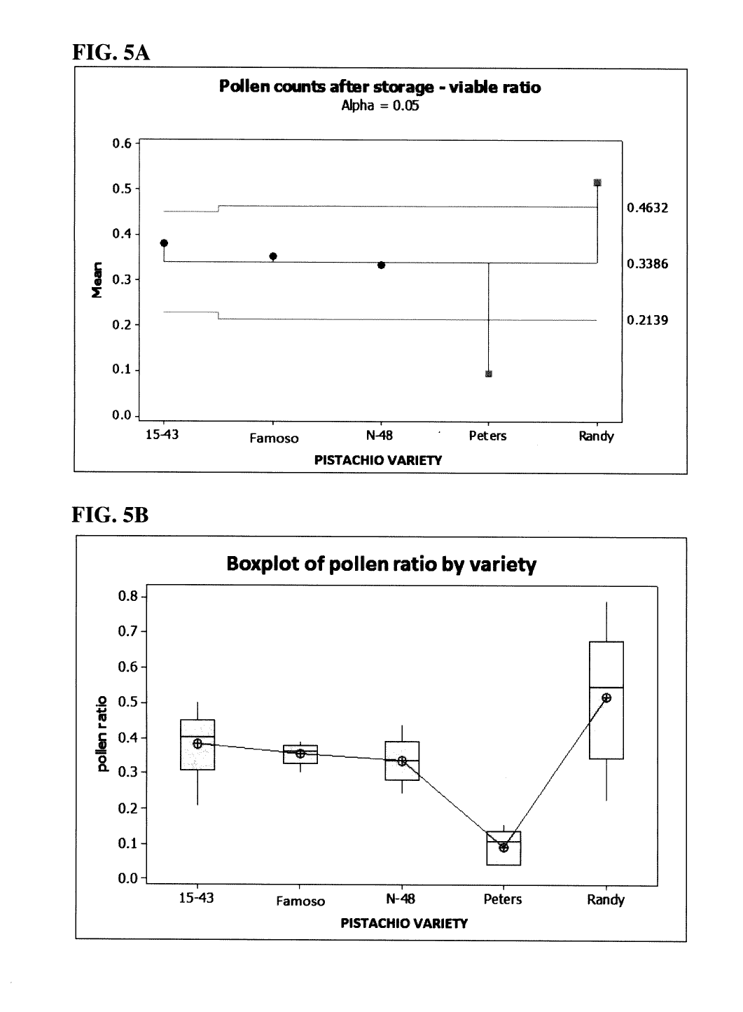

Aged Pollen Germination: In Year 26, pollen was collected from ‘Famoso’ and several comparison male pistachio varieties, including ‘Randy’ and ‘Peters’. The collected pollen was stored in a refrigerator for two days prior to conducting viable pollen counts (germination assay). This “aged pollen” count provides an estimate of pollen durability. Mean viable pollen counts for each variety are presented in FIG. 5A, and boxplots of these viable pollen ratios by variety are shown in FIG. 5B. Raw mean data and standard errors are presented in TABLE 4. Additional data and analysis are provided in TABLE 5A and TABLE 5B. Aged pollen for ‘Famoso’ was more viable than that of ‘Peters’, but less than that of ‘Randy’.

TABLE 4
“Aged Pollen” Viability Ratios

| | | Standard |
|---|---|---|
| | | Error |
| Variety | Mean | Mean |
| ‘15-43’ | 0.38198 | 0.04487 |
| ‘Famoso’ | 0.35480 | 0.04915 |
| ‘N-48’ | 0.33600 | 0.04915 |
| ‘Peters’ | 0.09402 | 0.04915 |
| ‘Randy’ | 0.51728 | 0.04915 |

TABLE 5A
Least Squares Means for germination of pollen in Year 26,
counted after 4° C. storage.

| Cultivar | Mean % | SE Mean % |
|---|---|---|
| N-48 | 33.60 | 4.915 |
| Peters | 9.40 | 4.915 |
| Randy | 51.73 | 4.915 |
| B15-43 | 38.20 | 4.487 |
| Famoso | 35.48 | 4.915 |

TABLE 5B
GLM ANOVA showing highly significant differences among cultivars.
GLM Analysis of Variance for Germination of Stored Pollen

| Source | DF | SS | MS | F | P |
|---|---|---|---|---|---|
| ACC | 4 | 0.47137 | 0.11784 | 9.76 | 0.000 |
| Error | 21 | 0.25368 | 0.01208 | | |
| Total | 25 | 0.72504 | | | |

S = 0.109908
R-Sq = 65.01%
R-Sq(adj) = 58.35%

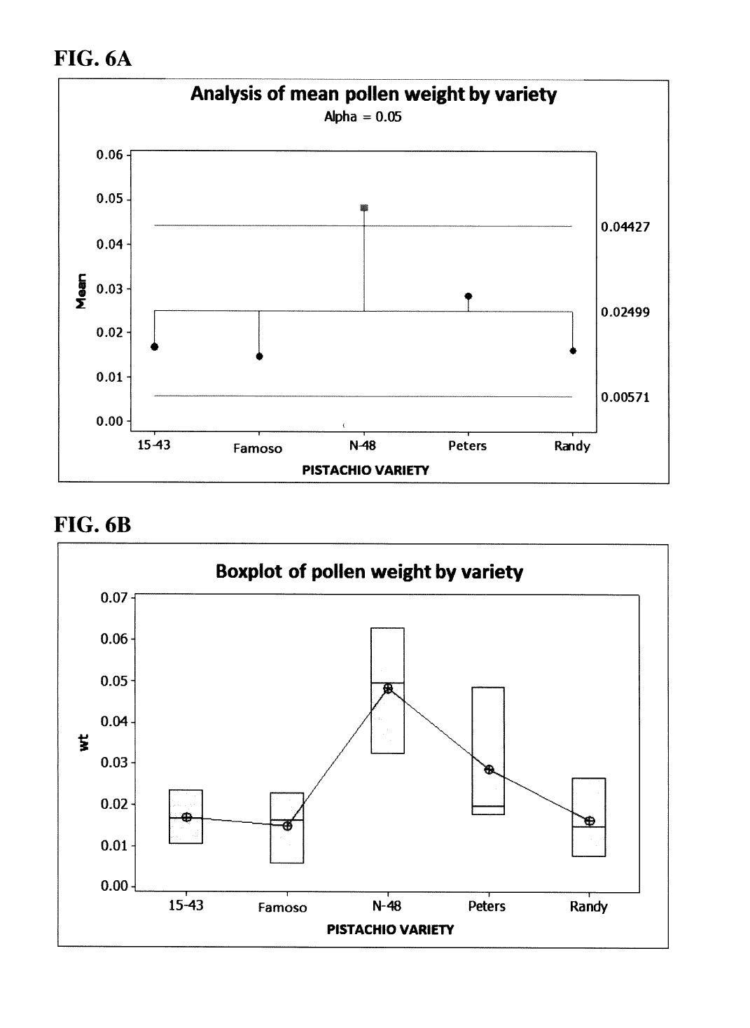

Pollen Quantities: During March of Year 26, branches with dehiscing inflorescences were collected and evaluated. Treatments involved taking four to five shoots that were 8-12 inches long with dehiscing inflorescences and placing them on craft paper overnight, followed by pollen collection the following morning. Pollen from three replicates of each treatment were collected and weighed. Treatments were normalized by counting the number of actively dehiscing inflorescences. ANOVA and ANOMA were performed with MiniTab. Mean pollen weights for each variety are presented in FIG. 6A, and boxplots of these viable pollen ratios by variety are shown in FIG. 6B. Raw mean data and standard deviations are presented in TABLE 6. Additional data and analysis are provided in TABLE 7A and TABLE 7B. ‘Famoso’ provides good quantities of viable pollen. Note that variety ‘15-43’ is also referred to as variety ‘B 15-43’.

TABLE 6
Pollen quantity (weight) in Year 26

| | | | Standard |
|---|---|---|---|
| Variety | N | Mean | Deviation |
| ‘15-43’ | 3 | 0.01683 | 0.00655 |
| ‘Famoso’ | 3 | 0.01487 | 0.00858 |
| ‘N-48’ | 3 | 0.04833 | 0.01524 |
| ‘Peters’ | 3 | 0.02863 | 0.01741 |
| ‘Randy’ | 3 | 0.01627 | 0.00958 |

TABLE 7A
Least Squares Means for pollen quantities (grams/inflorescence).

| Accession | Mean | SE Mean |
|---|---|---|
| N-48 | 0.04833 | 0.007042 |
| Peters | 0.02863 | 0.007042 |
| Randy | 0.01627 | 0.007042 |
| 15-43 | 0.01683 | 0.007042 |
| Famoso | 0.01487 | 0.007042 |

TABLE 7B
GLM ANOVA for pollen quantities per inflorescence.
GLM for pollen quantification

| Source | DF | SS | MS | F | P |
|---|---|---|---|---|---|
| Acc | 4 | 0.002410 | 0.000602 | 4.05 | 0.033 |
| Error | 10 | 0.001488 | 0.000149 | | |
| Total | 14 | 0.003898 | | | |

S = 0.01220
R-Sq = 61.83%
R-Sq(adj) = 46.56%

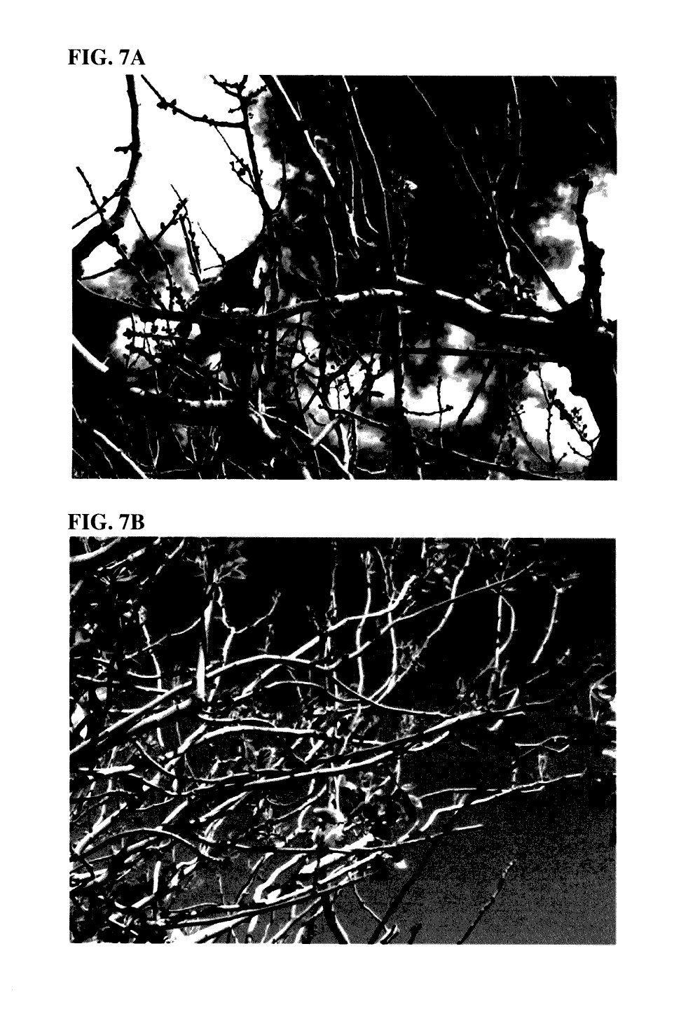

Inflorescence Density: The tree canopies of several male cultivars were photographed to provide an approximate evaluation of the number of inflorescences in the canopy. It is difficult to develop methods that accurately quantify this variable, which is highly dependent on tree size, pruning, and tree health. FIG. 7A and FIG. 7B provide a visual comparison of ‘Famoso’ and ‘Peters’, taken at their respective bloom periods. ‘Peters’ had very scattered bloom in Year 26, with many buds never breaking dormancy.

What is claimed is:

1. A new and distinct variety of pistachio tree designated ‘Famoso’ as shown and described herein.
